(19) United States
(12) Patent Application Publication
(10) Pub. No.: US 2025/0235445 A1
(43) Pub. Date: Jul. 24, 2025

(54) NOVEL PHARMACEUTICAL FORMULATION

(71) Applicant: RB HEALTH (US) LLC, Parsippany, NJ (US)

(72) Inventors: RAGHU CAVATUR, PARSIPPANY, NJ (US); KEVIN CHEN, PARSIPPANY, NJ (US); MATTHEW JAMES KASER, Parsippany, NJ (US); HONGCHUN QIU, Basking Ridge, NJ (US)

(21) Appl. No.: 19/027,993

(22) Filed: Jan. 17, 2025

Related U.S. Application Data
(63) Continuation of application No. 17/486,225, filed on Sep. 27, 2021.
Application No. 17/486,225 is a continuation of application No. 15/520,752, filed on Apr. 20, 2017, now abandoned.
(60) Provisional application No. 62/066,532, filed on Oct. 21, 2014.

(30) Foreign Application Priority Data
Apr. 21, 2015 (GB) 1506755.6

(51) Int. Cl. A61K 31/485; A61K 9/20; A61K 9/24; A61K 31/09; A61K 31/137; A61K 31/192; A61K 45/06; A61K 47/02; A61K 47/12; A61K 47/20; A61K 47/26; A61K 47/32; A61K 47/36; A61K 47/38

(52) U.S. Cl. CPC A61K 31/485; A61K 9/2054; A61K 9/209; A61K 31/09; A61K 31/137; A61K 31/192; A61K 45/06; A61K 47/02; A61K 47/12; A61K 47/20; A61K 47/26; A61K 47/32; A61K 47/36; A61K 47/38

(57) ABSTRACT

The present invention is directed to a pharmaceutical composition comprising a pharmaceutically effective amount of guaifenesin, naproxen and at least one further active which is selected to be an antitussive, a decongestant or an antihistamine.

This application is a continuation of U.S. patent application Ser. No. 17/486,225, filed Sep. 27, 2021, which is a continuation of U.S. application Ser. No. 15/520,752, filed Apr. 20, 2017, which is a national phase entry of PCT/GB2015/053151, filed on Oct. 21, 2015, which claims the benefit of British Application No. 1506755.6, filed Apr. 21, 2015, and the benefit of U.S. Provisional Patent Application No. 62/066,532, filed on Oct. 21, 2014, the contents of each of which are incorporated by reference herein in their entirety

### FIELD OF THE INVENTION

The present invention relates to a novel pharmaceutical formulation.

In particular, the present invention is directed to a sustained release formulation for oral administration comprising guaifenesin, naproxen and at least one additional active drug ingredient.

### RELATED ART

Pharmaceutical compositions comprising combinations of actives are well-known in the prior art. For example, many OTC cold & flu remedies include an anti-inflammatory together with an antitussive or cough suppressant. In addition, there are a number of pain killers which are based on a combination of one or more of ibuprofen, aspirin, paracetamol and codeine. There are also available immediate release products which combine more than two actives. For example, the TYLENOL® range includes products which combine paracetamol, dextromethorphan & guaifenesin. However, as these products are immediate release products re-dosing is required every 4 to 6 hours in order to maintain a therapeutic effect.

However, there is no specific teaching in the prior art of a sustained release dosage form containing guaifenesin, an analgesic and at least one other pharmaceutically active agent which is capable of sustaining a therapeutic effect for each of the actives for at least twelve hours.

Such a combination would be advantageous to develop as it would provide an individual relief from the symptoms of a cough, cold, or flu for an extended period of up to 12 hours. In particular, it would be desirable to develop a product which contains actives that can provide relief from the symptoms of a cough or cold as well as an analgesic. The low solubility of analgesics, however, can impact the dissolution of other active compounds in a combination product. This is particularly true in conventional extended release products where release control is dependent on the erosion of a polymer matrix.

In the area of solid single-dose combination products, such as tablets or caplets, most of the prior art is directed towards combinations of two different actives, each active being contained in a separate layer from the other with the third layer acting as a barrier to prevent any negative interactions between the active-containing layers.

WO 00/059479 discloses a novel pharmaceutical dosage form that provides pulsatile delivery of methylphenidate. WO 99/17745 describes a controlled-release monolithic system for oral administration comprising a disintegrating layer, an erodible layer and a swelling layer, of which two are external and one is intermediate, each layer containing one or more drugs.

### DETAILED DESCRIPTION OF EMBODIMENTS OF THE INVENTION

According to a first aspect of the present invention there is provided a pharmaceutical composition comprising a pharmaceutically effective amount of at least three different pharmaceutical actives selected from expectorant, an analgesic and at least one further active which is selected to be an antitussive, a decongestant or an antihistamine wherein the composition provides a therapeutic effect in respect of each active for a period of up to 12 hours.

Typically the analgesic is selected to be an NSAID. The NSAID can be selected from naproxen, ketoprofen, diclofenac, ibuprofen or flurbiprofen.

The expectorant can be guaifenesin.

The at least one other drug can be selected from an antitussive such as dextromethorphan, codeine, codeine phosphate, codeine sulphate, diphenhydramine citrate, and diphenhydramine hydrochloride, a decongestant such as phenylephrine hydrochloride, phenylpropanolamine hydrochloride, pseudoephedrine or ephedrine, an antihistamine such as chlorpheniramine maleate, brompheniramine maleate, phenindamine tartrate, pyrilamine maleate, doxylamine succinate, phenyltoloxamine citrate, diphenhydramine hydrochloride, promethazine, and clemastine fumerate, or a combination thereof. Preferred actives are dextromethorphan or pseudoephedrine.

The total amount of expectorant can be from about 200 mg-2400 mg, preferably about 600 mg-1200 mg. A preferred amount of expectorant is 600 mg. In an alternative embodiment, a preferred amount is 1200 mg.

The total amount of analgesic can be from about 5-4000 mg, preferably 110-220 mg. A preferred amount of analgesic is 110 mg. In an alternative embodiment, a preferred amount is 220 mg.

When the other drug is selected to be dextromethorphan, the total amount of said dextromethorphan can be from about 10-120 mg, preferably 15-60 mg. More preferably, the amount of dextromethorphan is 25-35 mg. A preferred amount of dextromethorphan is 30 mg. In an alternative embodiment, a preferred amount is 60 mg.

When the at least one other drug is selected to be pseudoephedrine, the total amount of said pseudoephedrine can be from about 10-240 mg, preferably 60-120 mg.

In a preferred combination of pharmaceutically active ingredients the expectorant is guaifenesin, the analgesic is naproxen and the antitussive is dextromethorphan.

The ratio of guaifenesin:naproxen:dextromethorphan can be from about 40:8:1 to about 10:3:1. A preferred ratio from 22:4:1 to 17:3:1. A most preferred ratio is 20:3.67:1.

The ratio of guaifensin:naproxen can be from about 1:1 to about 10:1, preferably from about 2:1 to about 7:1, more preferably from about 4:1 to about 6:1. A preferred ratio is about 5.45:1.

The ratio of the naproxen:dextromethorphan can be from about 1:1 to about 10:1, preferably from about 2:1 to about 7:1, more preferably from about 3:1 to about 5:1. A preferred ratio is about 3.67:1.

When an active other than dextromethorphan is used the ratio of naproxen: antitussive/decongestant/antihistamine can be from 10:1 to 2:1.

When flurbiprofen is selected to be the analgesic, the ratio of expectorant:analgesic:antitussive/decongestant/antihistamine can be from 25:5:1 to 5:0.5:1. The ratio of expectorant:analgesic can be from 10:1 to 5:1. The ratio of analgesic:antitussive/decongestant/antihistamine can be from 1:1.5-1:0.2.

When ibuprofen is selected to be the analgesic, the ratio of expectorant:analgesic:antitussive/decongestant/antihistamine can be from 25:30:1 to 5:5:1. The ratio of expectorant:analgesic can be from 1:1 to 1:5. The ratio of analgesic:antitussive/decongestant/antihistamine can be from 30:1-5:1.

When diclofenac is selected to be the analgesic, the ratio of expectorant:analgesic:antitussive/decongestant/antihistamine can be from 25:5:1 to 5:0.5:1. The ratio of expectorant:analgesic can be from 20:1 to 5:1. The ratio of analgesic:antitussive/decongestant/antihistamine can be from 1:2-1:0.2.

When ketoprofen is selected to be the analgesic, the ratio of expectorant:analgesic:antitussive/decongestant/antihistamine can be from 25:5:1 to 5:0.5:1. The ratio of expectorant:analgesic can be from 10:1 to 5:1. The ratio of analgesic:antitussive/decongestant/antihistamine can be from 1:1.5-1:0.2.

The composition can be in the form of one or more tablets, caplets, or capsules, gel, elixir, suspension, syrup, emulsion, powder, or granules. Typically, the composition is in the form of either a soft capsule or a hard gel capsule. When the composition is in the form of more than one tablets or caplets, the more than one tablets or caplets are contained within a single capsule. The capsule can be made of any suitable material, but is typically made of a gelatin material, hydroxyl propyl methyl cellulose or an alginate. The capsule can be in the form of either a soft capsule or a hard gel capsule.

The composition can comprise immediate and sustained release portions. As set forth herein, “portion” means a part of a whole, either separated or integrated with it. Thus, a product with two or more portions may have, but does not necessarily require, separate or discrete structural elements. As further set forth herein “sustained release” refers to a type of “modified release”, and these terms are used interchangeably throughout.

The analgesic can be incorporated into the composition such that it is the sole active in the portion in which it is contained.

Typically, the composition is provided with both sustained-release and immediate-release portions comprising the expectorant.

In a preferred embodiment the composition comprises a first immediate release portion which comprises guaifenesin and a decongestant or an antitussive, a second immediate release portion which comprises naproxen, and a sustained release portion which comprises guaifenesin and a decongestant or an antitussive. In further preferred embodiments the antitussive can be selected to be dextromethorphan or the decongestant can be selected to be pseudoephedrine.

According to a second aspect of the present invention there is provided a pharmaceutical composition comprising a pharmaceutically effective amount of guaifenesin, naproxen and at least one further active which is selected to be an antitussive, a decongestant or an antihistamine.

The at least one other drug can be selected from an antitussive such as dextromethorphan, codeine, codeine phosphate, codeine sulphate, diphenhydramine citrate, and diphenhydramine hydrochloride, a decongestant such as phenylephrine hydrochloride, phenylpropanolamine hydrochloride, pseudoephedrine or ephedrine, an antihistamine such as chlorpheniramine maleate, brompheniramine maleate, phenindamine tartrate, pyrilamine maleate, doxylamine succinate, phenyltoloxamine citrate, diphenhydramine hydrochloride, promethazine, and clemastine fumerate, or a combination thereof. Preferred actives are dextromethorphan or pseudoephedrine.

The total amount of guaifenesin can be from about 200 mg-2400 mg, preferably about 600 mg-1200 mg. A preferred amount of expectorant is 600 mg. In an alternative embodiment, a preferred amount is 1200 mg.

The total amount of naproxen can be from about 5-400 mg, preferably 110-220 mg. A preferred amount of analgesic is 110 mg. In an alternative embodiment, a preferred amount is 220 mg.

When the other drug is selected to be dextromethorphan, the total amount of said dextromethorphan can be from about 10-120 mg, preferably 15-60 mg. More preferably, the amount of dextromethorphan is 25-35 mg. A preferred amount of dextromethorphan is 30 mg. In an alternative embodiment, a preferred amount is 60 mg.

When the at least one other drug is selected to be pseudoephedrine, the total amount of said pseudoephedrine can be from about 10-240 mg, preferably 60-120 mg.

In a preferred combination of pharmaceutically active ingredients the expectorant is guaifenesin, the analgesic is naproxen and the antitussive is dextromethorphan.

The ratio of guaifenesin:naproxen:dextromethorphan can be from about 40:8:1 to about 10:3:1. A preferred ratio from 22:4:1 to 17:3:1. A most preferred ratio is 20:3.67:1.

The ratio of guaifensin:naproxen can be from about 1:1 to about 10:1, preferably from about 2:1 to about 7:1, more preferably from about 4:1 to about 6:1. A preferred ratio is about 5.45:1.

The ratio of the naproxen:dextromethorphan can be from about 1:1 to about 10:1, preferably from about 2:1 to about 7:1, more preferably from about 3:1 to about 5:1. A preferred ratio is about 3.67:1.

When an active other than dextromethorphan is used the ratio of naproxen: antitussive/decongestant/antihistamine can be from 10:1 to 2:1.

The composition can be in the form of one or more tablets, caplets, or capsules, gel, elixir, suspension, syrup, emulsion, powder, or granules. Typically, the composition is in the form of a capsule. When the composition is in the form of more than one tablets or caplets, the more than one tablets or caplets are contained within a single capsule. The capsule can be made of any suitable material, but is typically made of a gelatin material.

The composition can comprise immediate and sustained release portions.

Typically, the composition is provided with both sustained-release and immediate-release portions comprising the expectorant.

The naproxen can be incorporated into the composition such that it is the sole active in the portion in which it is contained.

Typically, the composition is provided with both sustained-release and immediate-release portions comprising guaifenesin.

In a preferred embodiment the composition comprises a first immediate release portion which comprises guaifenesin and a decongestant or an antitussive, a second immediate release portion which comprises naproxen, and a sustained release portion which comprises guaifenesin. In further preferred embodiments the antitussive can be selected to be dextromethorphan or the decongestant can be selected to be pseudoephedrine.

The composition can be in the form of a tablet, caplet, capsule, gel, elixir, suspension, syrup or emulsion. Typically, the composition is in the form of a capsule.

In a particularly preferred embodiment of the composition of the first and second embodiments, the composition comprises guaifenesin, naproxen and dextromethorphan.

The composition according to either of the first or second aspect can comprise:

| | |
|---|---|
| (a) 50-85% | Guaifenesin; |
| (b) up to 5% | Dextromethorphan or a pharmaceutically acceptable salt |
| | thereof; and |
| (c) 5-30% | Naproxen or a pharmaceutically acceptable salt thereof; |

The composition according to either of the first or second aspect can comprise:

| | |
|---|---|
| (a) 55-65% | Guaifenesin; |
| (b) 1-5% | Dextromethorphan or a pharmaceutically acceptable salt |
| | thereof; |
| (c) 5-15% | Naproxen or a pharmaceutically acceptable salt thereof; |
| (d) 1-10% | Controlled release polymers; |
| (e) 0.1-10% | Binders; |
| (f) 0.1-5% | Disintegrants; |
| (g) 5-25% | Diluents; and |
| (h) up to 1% | Lubricants. |

A preferred embodiment of the composition according to either of the first or second aspect can comprise:

| | |
|---|---|
| (a) 55-65% | Guaifenesin; |
| (b) 1-5% | Dextromethorphan or a pharmaceutically acceptable salt |
| | thereof; |
| (c) 5-15% | Naproxen or a pharmaceutically acceptable salt thereof; |
| (d) 1-8% | Hypromellose; |
| (e) 10-25% | Microcrystalline cellulose; |
| (f) 0.1to 2.5% | Povidone; |
| (g) 0.1to 4.0% | Croscarmellose Sodium; |
| (h) 0.1to 2.0% | Hydroxy Ethyl Cellulose; and |
| (i) up to 1% | Magnesium stearate. |

An alternative preferred embodiment can comprise:

| | |
|---|---|
| (a) 58-63% | Guaifenesin; |
| (b) 2-3.5% | Dextromethorphan or a pharmaceutically acceptable salt |
| | thereof; |
| (c) 10-12% | Naproxen or a pharmaceutically acceptable salt thereof; |
| (d) 1-2% | Hypromellose; |
| (e) 15-25% | Microcrystalline cellulose; |
| (f) 1-2% | Povidone; |
| (g) 0.5-2.5% | Croscarmellose sodium; |
| (h) 0.5 to 1% | Hydroxyethyl cellulose; and |
| (i) up to 0.5% | Magnesium stearate. |

In an alternative embodiment the composition can comprise:

| | |
|---|---|
| (a) 55-65% | Guaifenesin; |
| (b) 1-5% | Dextromethorphan or a pharmaceutically |
| | acceptable salt thereof; |
| (c) 5-15% | Naproxen or a pharmaceutically acceptable |
| | salt thereof; |
| (d) 1-8% | Hypromellose; |
| (e) 10-25% | Microcrystalline cellulose; |
| (f) 0.1 to 2.5% | Povidone; |
| (g) 0.1to 2.0% | Carbomer; |
| (h) 0.1 2.0% | Sodium Starch Glycolate; |
| (i) up to 1% | Magnesium stearate; and |

An alternative preferred embodiment can comprise:

| | |
|---|---|
| (a) 59-65% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1-6% | Hypromellose; |
| (e) 10-20% | Microcrystalline cellulose; |
| (f) 0.3 to 1% | Povidone; |
| (g) 0.5-1% | Carbomer; |
| (h) 0.1 to 0.5% | Sodium Starch Glycolate; and |
| (i) up to 1% | Magnesium stearate. |

An alternative preferred embodiment can comprise:

| | |
|---|---|
| (a) 59-65% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1-6% | Hypromellose; |
| (e) 10-20% | Microcrystalline cellulose; |
| (f) 0.3 to 1% | Povidone; |
| (g) 0.5-1% | Carbomer; |
| (h) 1-2% | Croscarmellose sodium; |
| (i) 0.1 to 0.5% | Sodium Starch Glycolate; and |
| (j) up to 1% | Magnesium stearate. |

An alternative preferred embodiment of the composition according to either of the first or second aspect can comprise:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 3-5% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; and |
| (i) 0.25-0.5% | Sodium starch glycolate. |

The composition can further comprise one or more of:

| | |
|---|---|
| (j) 0.5-1.5% | Lactose; |
| (k) 2.5-3.5% | Sodium lauryl sulphate; and |
| (l) 0.1-1.5% | Croscarmellose sodium. |

An alternative preferred embodiment of the composition according to either of the first or second aspect can comprise:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 0.1-0.6% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; and |
| (i) 0.25-0.5% | Sodium starch glycolate. |

The composition can further comprise one or more of:

| | |
|---|---|
| (j) 0.5-1.5% | Lactose; |
| (k) 2.5-3.5% | Sodium lauryl sulphate; and |
| (l) 0.1-1.5% | Croscarmellose sodium. |

An alternative preferred embodiment of the composition according to either of the first or second aspect can comprise:

| | |
|---|---|
| (a) 63-66% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1.75-3.0% | Hypromellose; |
| (e) 11-13% | Microcrystalline cellulose; |
| (f) 1-2% | Povidone; |
| (g) 0.5-0.75% | Croscarmellose Sodium; |
| (h) 1-2% | Carbomer; |
| (i) 0.1-0.5% | Magnesium stearate; |
| (j) 0.5-1.5% | Lactose; and optionally |
| (k) 2-3% | Sodium Lauryl Sulphate. |

An alternative preferred embodiment of the composition according to either of the first or second aspect can comprise:

| | |
|---|---|
| (a) 64-66% | Guaifenesin; |
| (b) 3-5% | Dextromethorphan or a pharmaceutically |
| | acceptable salt thereof; |
| (c) 11-13% | Naproxen or a pharmaceutically acceptable |
| | salt thereof; |
| (d) 1.75-3.0% | Hypromellose; |
| (e) 6-8.5% | Microcrystalline cellulose; |
| (f) 1 -2% | Povidone; |
| (g) 1-5% | Croscarmellose Sodium; |
| (h) 0.5-1% | Carbomer; |
| (i) 0.5-1% | Magnesium stearate; and |
| (j) 0.5-1% | Crospovidone. |

The composition can further include one or more of:

| | |
|---|---|
| (k) 2-3% | Sodium Lauryl Sulphate; and |
| (l) 6-7% | Sodium Bicarbonate. |

An alternative preferred embodiment of the composition according to either of the first or second aspect can comprise:

| | |
|---|---|
| (a) 64-66% | Guaifenesin; |
| (b) 3-5% | Dextromethorphan or a pharmaceutically |
| | acceptable salt thereof; |
| (c) 11-13% | Naproxen or a pharmaceutically acceptable |
| | salt thereof; |
| (d) 1.75-3.0% | Hypromellose; |
| (e) 6-8.5% | Microcrystalline cellulose; |
| (f) 1-2% | Povidone; |
| (g) 1-5% | Croscarmellose Sodium; |
| (h) 0.5-1% | Carbomer; and |
| (i) 0.5-1% | Magnesium stearate. |

An alternative preferred embodiment of the composition according to either of the first or second aspect can comprise:

| | |
|---|---|
| (a) 62-65% | Guaifenesin; |
| (b) 3-5% | Dextromethorphan or a pharmaceutically |
| | acceptable salt thereof; |
| (c) 11-13% | Naproxen or a pharmaceutically acceptable |
| | salt thereof; |
| (d) 1.75-3.0% | Hypromellose; |
| (e) 6-8.5% | Microcrystalline cellulose; |
| (f) 1-2% | Povidone; |
| (g) 1-5% | Croscarmellose Sodium; |
| (h) 0.5-1% | Carbomer; and |
| (i) 0.5-1% | Magnesium stearate; |
| (j) 2-3% | Sodium Lauryl Sulphate; and |
| (k) 5-8% | Sodium Bicarbonate. |

An alternative preferred embodiment of the composition according to either of the first or second aspect can comprise:

| | |
|---|---|
| (a) 58-60% | Guaifenesin; |
| (b) 2-3% | Dextromethorphan or a pharmaceutically |
| | acceptable salt thereof; |
| (c) 10-11% | Naproxen or a pharmaceutically acceptable |
| | salt thereof; |
| (d) 2-3% | Hypromellose; |
| (e) 7-8% | Microcrystalline cellulose; |
| (f) 1-2% | Povidone; |
| (g) 2-3% | Croscarmellose Sodium; |
| (h) 0.1-0.2% | Magnesium stearate; |
| (i) 0.5-1% | Crospovidone; |
| (j) 1-3% | Hydroxyethyl cellulose; |
| (k) 2-3% | Sodium Lauryl Sulphate; and |
| (l) 9-10% | Sodium bicarbonate. |

An alternative preferred embodiment of the composition according to either of the first or second aspect can comprise:

| | |
|---|---|
| (a) 64-66% | Guaifenesin; |
| (b) 3-5% | Dextromethorphan or a pharmaceutically |
| | acceptable salt thereof; |
| (c) 11-13% | Naproxen or a pharmaceutically acceptable |
| | salt thereof; |
| (d) 2-3% | Hypromellose; |
| (e) 6-7% | Microcrystalline cellulose; |
| (f) 1-2% | Povidone; |
| (g) 0.1-0.5% | Magnesium stearate; |
| (h) 7-10% | Crospovidone; and |
| (i) 1-3% | Hydroxyethyl cellulose. |

A preferred embodiment of the composition according to either of the first or second aspect consists essentially of:

| | |
|---|---|
| (a) 55-65% | Guaifenesin; |
| (b) 1-5% | Dextromethorphan or a pharmaceutically |
| | acceptable salt thereof; |
| (c) 5-15% | Naproxen or a pharmaceutically acceptable |
| | salt thereof; |
| (d) 1-8% | Hypromellose; |
| (e) 10-25% | Microcrystalline cellulose; |
| (f) 0.1to 2.5% | Povidone; |
| (g) 0.1to 4.0% | Croscarmellose Sodium; |
| (h) 0.1to 2.0% | Hydroxy Ethyl Cellulose; and |
| (i) up to 1% | Magnesium stearate. |

An alternative preferred embodiment consists essentially of:

| | |
|---|---|
| (a) 58-63% | Guaifenesin; |
| (b) 2 -3.5% | Dextromethorphan or a pharmaceutically |
| | acceptable salt thereof; |
| (c) 10-12% | Naproxen or a pharmaceutically acceptable |
| | salt thereof; |
| (d) 1-2% | Hypromellose; |
| (e) 15-25% | Microcrystalline cellulose; |
| (f) 1-2% | Povidone; |
| (g) 0.5-2.5% | Croscarmellose sodium; |
| (h) 0.5 to 1% | Hydroxyethyl cellulose; and |
| (i) up to 0.5% | Magnesium stearate. |

In an alternative embodiment the composition consists essentially of:

| | |
|---|---|
| (a) 55-65% | Guaifenesin; |
| (b) 1-5% | Dextromethorphan or a pharmaceutically |
| | acceptable salt thereof; |
| (c) 5-15% | Naproxen or a pharmaceutically acceptable |
| | salt thereof; |
| (d) 1-8% | Hypromellose; |
| (e) 10-25% | Microcrystalline cellulose; |
| (f) 0.1 to 2.5% | Povidone; |
| (g) 0.1to 2.0% | Carbomer; |
| (h) 0.12.0% | Sodium Starch Glycolate; |
| (i) up to 1% | Magnesium stearate; and |

An alternative preferred embodiment consists essentially of:

| | |
|---|---|
| (a) 59-65% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1-6% | Hypromellose; |
| (e) 10-20% | Microcrystalline cellulose; |
| (f) 0.3 to 1% | Povidone; |
| (g) 0.5-1% | Carbomer; |
| (h) 0.1 to 0.5% | Sodium Starch Glycolate; and |
| (i) up to 1% | Magnesium stearate. |

An alternative preferred embodiment consists essentially of:

| | |
|---|---|
| (a) 59-65% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1-6% | Hypromellose; |
| (e) 10-20% | Microcrystalline cellulose; |
| (f) 0.3 to 1% | Povidone; |
| (g) 0.5-1% | Carbomer; |
| (h) 1-2% | Croscarmellose sodium; |
| (i) 0.1 to 0.5% | Sodium Starch Glycolate; and |
| (j) up to 1% | Magnesium stearate. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 3-5% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; and |
| (i) 0.25-0.5% | Sodium starch glycolate. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| (c) 12-15% | acceptable salt thereof; |
| | Naproxen or a pharmaceutically acceptable |
| | salt thereof; |
| (d) 0.1-0.5% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; and |
| (i) 0.25-0.5% | Sodium starch glycolate. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 3-5% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; |
| (i) 0.25-0.5% | Sodium starch glycolate; |
| (j) 0.5-1.5% | Lactose; and |
| (k) 0.1-1.5% | Croscarmellose sodium. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| | thereof; |
| (c) 12-15% | Naproxen or a pharmaceutically |
| (d) 0.1-0.6% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; |
| (i) 0.25-0.5% | Sodium starch glycolate; |
| (j) 0.5-1.5% | Lactose; and |
| (k) 0.1-1.5% | Croscarmellose sodium. |

An alternative preferred embodiment of the composition according to either of the first or second aspect consists essentially of:

| | |
|---|---|
| (a) 63-66% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1.75-3.0% | Hypromellose; |
| (e) 11-13% | Microcrystalline cellulose; |
| (f) 1-2% | Povidone; |
| (g) 0.5-0.75% | Croscarmellose Sodium; |
| (h) 1-2% | Carbomer; |
| (i) 0.1-0.5% | Magnesium stearate; and |
| (j) 0.5-1.5% | Lactose. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 3-5% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; |
| (i) 0.25-0.5% | Sodium starch glycolate; |
| (j) 2-3.5% | Sodium Lauryl Sulphate; and |
| (k) 0.1-1.5% | Croscarmellose sodium. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 0.1-0.6% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; |
| (i) 0.25-0.5% | Sodium starch glycolate; |
| (j) 2-3.5% | Sodium Lauryl Sulphate; and |
| (k) 0.1-1.5% | Croscarmellose sodium. |

An alternative preferred embodiment of the composition according to either of the first or second aspect consists essentially of:

| | |
|---|---|
| (a) 64-69% | Guaifenesin; |
| (b) 3-5% | Dextromethorphan or a pharmaceutically |
| | acceptable salt thereof; |
| (c) 12-15% | Naproxen or a pharmaceutically acceptable |
| | salt thereof; |
| (d) 0.1-0.6% | Hypromellose; |
| (e) 9-15% | Microcrystalline cellulose; |
| (f) 0.4-0.65% | Povidone; |
| (g) 0.75-1.3% | Carbomer; |
| (h) 0.5-1% | Magnesium stearate; |
| (i) 0.25-0.5% | Sodium starch glycolate; |
| (j) 2-3.5% | Sodium Lauryl Sulphate; and |
| (k) 0.1-1.5% | Croscarmellose sodium. |

An alternative preferred embodiment of the composition according to either of the first or second aspect consists essentially of:

| | |
|---|---|
| (a) 63-66% | Guaifenesin; |
| (b) 3-5% | Dextromethorphan or a pharmaceutically |
| | acceptable salt thereof; |
| (c) 10-12% | Naproxen or a pharmaceutically acceptable |
| | salt thereof; |
| (d) 1.75-3.0% | Hypromellose; |
| (e) 11-13% | Microcrystalline cellulose; |
| (f) 1-2% | Povidone; |
| (g) 0.5-0.75% | Croscarmellose Sodium; |
| (h) 1-2% | Carbomer; |
| (i) 0.1-0.5% | Magnesium stearate; |
| (j) 0.5-1.5% | Lactose; and |
| (k) 2-3% | Sodium Lauryl Sulphate. |

An alternative preferred embodiment of the composition according to either of the first or second aspect consists essentially of:

| | |
|---|---|
| (a) 64-66% | Guaifenesin; |
| (b) 3-5% | Dextromethorphan or a pharmaceutically |
| | acceptable salt thereof; |
| (c) 11-13% | Naproxen or a pharmaceutically acceptable |
| | salt thereof; |
| (d) 1.75-3.0% | Hypromellose; |
| (e) 6-8.5% | Microcrystalline cellulose; |
| (f) 1-2% | Povidone; |
| (g) 1-5% | Croscarmellose Sodium; |
| (h) 0.5-1% | Carbomer; |
| (i) 0.5-1% | Magnesium stearate; |
| (j) 0.5-1% | Crospovidone; and |
| (k) 2-3% | Hypromellose. |

An alternative preferred embodiment of the composition according to either of the first or second aspect consists essentially of:

| | |
|---|---|
| (a) 64-66% | Guaifenesin; |
| (b) 3-5% | Dextromethorphan or a pharmaceutically |
| | acceptable salt thereof; |
| (c) 11-13% | Naproxen or a pharmaceutically acceptable |
| | salt thereof; |
| (d) 1.75-3.0% | Hypromellose; |
| (e) 6-8.5% | Microcrystalline cellulose; |
| (f) 1-2% | Povidone; |
| (g) 1-5% | Croscarmellose Sodium; |
| (h) 0.5-1% | Carbomer; |
| (i) 0.5-1% | Magnesium stearate; |
| (j) 0.5-1% | Crospovidone; |
| (k) 2-3% | Hypromellose; |
| (l) 2-3% | Sodium Lauryl Sulphate; and |
| (m) 6-7% | Sodium Bicarbonate. |

An alternative preferred embodiment of the composition according to either of the first or second aspect consists essentially of:

| | | |
|---|---|---|
| (a) | 58-60% | Guaifenesin; |
| (b) | 2-3% | Dextromethorphan or a pharmaceutically acceptable |
| | | salt thereof; |
| (c) | 10-11% | Naproxen or a pharmaceutically acceptable salt |
| | | thereof; |
| (d) | 2-3% | Hypromellose; |
| (e) | 7-8% | Microcrystalline cellulose; |
| (f) | 1-2% | Povidone; |
| (g) | 2-3% | Croscarmellose Sodium; |
| (h) | 0.1-0.2% | Magnesium stearate; |
| (i) | 0.5-1% | Crospovidone; |
| (j) | 1-3% | Hydroxyethyl cellulose; |
| (k) | 2-3% | Sodium Lauryl Sulphate; and |
| (l) | 9-10% | Sodium bicarbonate. |

An alternative preferred embodiment of the composition according to either of the first or second aspect consists essentially of:

| | | |
|---|---|---|
| (a) | 64-66% | Guaifenesin; |
| (b) | 3-5% | Dextromethorphan or a pharmaceutically acceptable |
| | | salt thereof; |
| (c) | 11-13% | Naproxen or a pharmaceutically acceptable |
| | | salt thereof; |
| (d) | 2-3% | Hypromellose; |
| (e) | 6-7% | Microcrystalline cellulose; |
| (f) | 1-2% | Povidone; |
| (g) | 0.1-0.5% | Magnesium stearate; |
| (h) | 7-10% | Crospovidone; and |
| (i) | 1-3% | Hydroxyethyl cellulose. |

An alternative preferred embodiment of the composition according to either of the first or second aspect consists essentially of:

| | | |
|---|---|---|
| (a) | 62-65% | Guaifenesin; |
| (b) | 3-5% | Dextromethorphan or a pharmaceutically acceptable |
| | | salt thereof; |
| (c) | 11-13% | Naproxen or a pharmaceutically acceptable |
| | | salt thereof; |
| (d) | 1.75-3.0% | Hypromellose; |
| (e) | 6-8.5% | Microcrystalline cellulose; |
| (f) | 1-2% | Povidone; |
| (g) | 1-5% | Croscarmellose Sodium; |
| (h) | 0.5-1% | Carbomer; and |
| (i) | 0.5-1% | Magnesium stearate; |
| (j) | 2-3% | Sodium Lauryl Sulphate; and |
| (k) | 5-8% | Sodium Bicarbonate. |

An alternative preferred embodiment of the composition according to either of the first or second aspect consists essentially of:

| | | |
|---|---|---|
| (a) | 64-66% | Guaifenesin; |
| (b) | 3-5% | Dextromethorphan or a pharmaceutically acceptable |
| | | salt thereof; |
| (c) | 11-13% | Naproxen or a pharmaceutically acceptable |
| | | salt thereof; |
| (d) | 1.75-3.0% | Hypromellose; |
| (e) | 6-8.5% | Microcrystalline cellulose; |
| (f) | 1-2% | Povidone; |
| (g) | 1-5% | Croscarmellose Sodium; |
| (h) | 0.5-1% | Carbomer; and |
| (i) | 0.5-1% | Magnesium stearate. |

According to a third aspect of the present invention there is provided a pharmaceutical dosage form which comprises at least 3 distinct portions wherein 2 of the portions have immediate release characteristics and a 3^{rd} has modified release characteristics wherein one of the immediate release portions comprises an expectorant and a second active which is an antitussive or a decongestant and the second immediate release portion comprises an analgesic having a half-life of twelve hours and wherein the modified release portion comprises guaifenesin and a second active which is an antitussive or a decongestant.

The dosage form can comprise a first composition which comprises both immediate and modified release portions of the expectorant and the second active which is an antitussive or a decongestant and a second composition which comprises an analgesic having a therapeutic effect of twelve hours.

The analgesic can be incorporated into the composition such that it is the sole active in the portion in which it is contained.

In preferred embodiments of the compositions of the first, second and third aspects of the present invention the analgesic-containing immediate release portion can comprise:

| | | |
|---|---|---|
| (a) | 50-52% | Naproxen or a pharmaceutically acceptable salt thereof; |
| (b) | 35-40% | Microcrystalline cellulose; |
| (c) | up to 5% | Povidone; |
| (d) | 5-8% | Croscarmellose sodium; and |
| (e) | up to 1% | Magnesium stearate. |

In alternative preferred embodiments of the compositions of the first, second and third aspects of the present invention the analgesic-containing immediate release portion can comprise:

| | | |
|---|---|---|
| (a) | 70-75% | Naproxen or a pharmaceutically acceptable salt thereof; |
| (b) | 20-25% | Microcrystalline cellulose; |
| (c) | up to 5% | Povidone; |
| (d) | up to 1% | Magnesium stearate. |

In further alternative preferred embodiments of the compositions of the first, second and third aspects of the present invention the analgesic-containing immediate release portion can comprise:

| | | |
|---|---|---|
| (a) | 70-75% | Naproxen or a pharmaceutically acceptable salt thereof; |
| (b) | 5-10% | Microcrystalline cellulose; |
| (c) | up to 5% | Povidone; |
| (d) | up to 1% | Magnesium stearate; |
| (e) | 5-10% | Lactose; and |
| (f) | up to 5% | Croscarmellose sodium. |

In further alternative preferred embodiments of the compositions of the first, second and third aspects of the present invention the analgesic-containing immediate release portion can comprise:

| | | |
|---|---|---|
| (a) | 70-75% | Naproxen or a pharmaceutically acceptable salt thereof; |
| (b) | 5-10% | Microcrystalline cellulose; |
| (c) | up to 5% | Povidone; |
| (d) | up to 1% | Magnesium stearate; |
| (e) | 5-10% | Lactose; and |
| (f) | 5-10% | Croscarmellose sodium. |

In further alternative preferred embodiments of the compositions of the first, second and third aspects of the present invention the analgesic-containing immediate release portion can comprise:

| | | |
|---|---|---|
| (a) | 70-75% | Naproxen or a pharmaceutically acceptable salt thereof; |
| (b) | 5-10% | Microcrystalline cellulose; |
| (c) | up to 5% | Povidone; |
| (d) | up to 1% | Magnesium stearate; |
| (e) | up to 5% | Lactose; and |
| (f) | 15-20% | Sodium Lauryl Sulphate. |

In further alternative preferred embodiments of the compositions of the first, second and third aspects of the present invention the analgesic-containing immediate release portion can comprise:

| | | |
|---|---|---|
| (a) | 70-75% | Naproxen or a pharmaceutically acceptable salt thereof; |
| (b) | 5-10% | Microcrystalline cellulose; |
| (c) | up to 5% | Povidone; |
| (d) | up to 1% | Magnesium stearate; |
| (e) | up to 2% | Croscarmellose sodium; and |
| (f) | 15-20% | Sodium Lauryl Sulphate. |

In preferred embodiments of the compositions of the first, second and third aspects of the present invention the guaifenesin-containing immediate release portion can comprise:

| | | |
|---|---|---|
| (a) | 30-40% | Guaifenesin; |
| (b) | up to 5% | Dextromethorphan or a pharmaceutically acceptable salt |
| | | thereof; |
| (c) | 50-60% | Microcrystalline cellulose; |
| (d) | up to 7% | Povidone; |
| (e) | up to 7% | Croscarmellose sodium; and |
| (f) | up to 1% | Magnesium stearate. |

In alternative preferred embodiments of the compositions of the first, second and third aspects of the present invention preferred embodiments of the compositions of the present the guaifenesin-containing immediate release portion can comprise:

| | | |
|---|---|---|
| (a) | 40-50% | Guaifenesin; |
| (b) | up to 5% | Dextromethorphan or a pharmaceutically acceptable salt |
| | | thereof; |
| (c) | 40-50% | Microcrystalline cellulose; |
| (d) | up to 5% | Hypromellose; |
| (e) | up to 5% | Sodium starch glycolate; and |
| (f) | up to 1% | Magnesium stearate. |

In alternative preferred embodiments of the compositions of the first, second and third aspects of the present invention preferred embodiments of the compositions of the present the guaifenesin-containing immediate release portion can comprise:

| | | |
|---|---|---|
| (a) | 50-65% | Guaifenesin; |
| (b) | up to 5% | Dextromethorphan or a pharmaceutically acceptable salt |
| | | thereof; |
| (c) | 25-40% | Microcrystalline cellulose; |
| (d) | up to 5% | Povidone; |
| (e) | up to 5% | Croscarmellose sodium; and |
| (f) | up to 1% | Magnesium stearate. |

In preferred embodiments of the compositions of the first, second and third aspects of the present invention the modified release portion can comprise:

| | | |
|---|---|---|
| (a) | 80-90% | Guaifenesin; |
| (b) | up to 5% | Dextromethorphan or a pharmaceutically acceptable |
| | | salt thereof; |
| (c) | up to 10% | Hypromellose; |
| (d) | up to 5% | Carbomer; |
| (e) | up to 1.55% | Magnesium stearate. |

In alternative preferred embodiments of the compositions of the first, second and third aspects of the present invention preferred embodiments of the compositions of the present invention the modified release portion can comprise:

| | | |
|---|---|---|
| (a) | 80-90% | Guaifenesin; |
| (b) | 3-6% | Dextromethorphan or a pharmaceutically acceptable salt |
| | | thereof; |
| (c) | up to 5% | Hypromellose; |
| (d) | up to 2% | Hydroxy ethylcellulose; |
| (e) | up to 5% | Microcrystalline cellulose; |
| (f) | up to 1% | Magnesium stearate. |

In alternative preferred embodiments of the compositions of the first, second and third aspects of the present invention preferred embodiments of the compositions of the present invention the modified release portion can comprise:

| | |
|---|---|
| (a) 80-90% | Guaifenesin; |
| (b) 3-6% | Dextromethorphan or a pharmaceutically acceptable salt |
| | thereof; |
| (c) up to 5% | Hypromellose; |
| (d) up to 5% | Hydroxy ethylcellulose; |
| (e) up to 5% | Microcrystalline cellulose; |
| (f) up to 1% | Magnesium stearate. |

In preferred embodiments of the compositions of the first, second and third aspects of the present invention the modified release portion can comprise:

| | |
|---|---|
| (a) 85-90% | Guaifenesin; |
| (b) 4-5% | Dextromethorphan or a pharmaceutically acceptable salt |
| | thereof; |
| (c) 3-6% | Hypromellose; |
| (d) 1-3% | Carbomer; |
| (e) 0.5-1% | Magnesium stearate. |

In preferred embodiments of the compositions of the first, second and third aspects of the present invention the modified release portion can comprise:

| | |
|---|---|
| (a) 90-93% | Guaifenesin; |
| (b) 4-6% | Dextromethorphan or a pharmaceutically acceptable salt |
| | thereof; |
| (c) 1-3% | Carbomer; |
| (d) 0.5-1% | Magnesium stearate. |

In preferred embodiments of the compositions of the first, second and third aspects of the present invention the analgesic-containing immediate release portion consists essentially of:

| | |
|---|---|
| (a) 50-52% | Naproxen or a pharmaceutically acceptable salt thereof; |
| (b) 35-40% | Microcrystalline cellulose; |
| (c) up to 5% | Povidone; |
| (d) 5-8% | Croscarmellose sodium; and |
| (e) up to 1% | Magnesium stearate. |

In alternative preferred embodiments of the compositions of the first, second and third aspects of the present invention the analgesic-containing immediate release portion consists essentially of:

| | |
|---|---|
| (a) 70-75% | Naproxen or a pharmaceutically acceptable salt thereof; |
| (b) 20-25% | Microcrystalline cellulose; |
| (c) up to 5% | Povidone; |
| (d) up to 1% | Magnesium stearate. |

In further alternative preferred embodiments of the compositions of the first, second and third aspects of the present invention the analgesic-containing immediate release portion consists essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) 5-10% | Lactose; and |
| (f) up to 5% | Croscarmellose sodium. |

In further alternative preferred embodiments of the compositions of the first, second and third aspects of the present invention the analgesic-containing immediate release portion consists essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) 5-10% | Lactose; and |
| (f) 5-10% | Croscarmellose sodium. |

In further alternative preferred embodiments of the compositions of the first, second and third aspects of the present invention the analgesic-containing immediate release portion consists essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) up to 5% | Lactose; and |
| (f) 15-20% | Sodium Lauryl Sulphate. |

In further alternative preferred embodiments of the compositions of the first, second and third aspects of the present invention the analgesic-containing immediate release portion consists essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) up to 2% | Croscarmellose sodium; and |
| (f) 15-20% | Sodium Lauryl Sulphate. |

In preferred embodiments of the compositions of the first, second and third aspects of the present invention the guaifenesin-containing immediate release portion consists essentially of:

| | |
|---|---|
| (a) 30-40% | Guaifenesin; |
| (b) up to 5% | Dextromethorphan or a pharmaceutically acceptable salt |
| | thereof; |
| (c) 50-60% | Microcrystalline cellulose; |
| (d) up to 7% | Povidone; |
| (e) up to 7% | Croscarmellose sodium; and |
| (f) up to 1% | Magnesium stearate. |

In alternative preferred embodiments of the compositions of the first, second and third aspects of the present invention preferred embodiments of the compositions of the present the guaifenesin-containing immediate release portion consists essentially of:

| | |
|---|---|
| (a) 40-50% | Guaifenesin; |
| (b) up to 5% | Dextromethorphan or a pharmaceutically acceptable salt |
| | thereof; |
| (c) 40-50% | Microcrystalline cellulose; |
| (d) up to 5% | Hypromellose; |
| (e) up to 5% | Sodium starch glycolate; and |
| (f) up to 1% | Magnesium stearate. |

In alternative preferred embodiments of the compositions of the first, second and third aspects of the present invention preferred embodiments of the compositions of the present the guaifenesin-containing immediate release portion consists essentially of:

| | |
|---|---|
| (a) 50-65% | Guaifenesin; |
| (b) up to 5% | Dextromethorphan or a pharmaceutically acceptable salt |
| | thereof; |
| (c) 25-40% | Microcrystalline cellulose; |
| (d) up to 5% | Povidone; |
| (e) up to 5% | Croscarmellose sodium; and |
| (f) up to 1% | Magnesium stearate. |

In preferred embodiments of the compositions of the first, second and third aspects of the present invention the modified release portion consists essentially of:

| | |
|---|---|
| (a) 80-90% | Guaifenesin; |
| (b) up to 5% | Dextromethorphan or a pharmaceutically acceptable salt |
| | thereof; |
| (c) up to 10% | Hypromellose; |
| (d) up to 5% | Carbomer; |
| (e) up to 1.55% | Magnesium stearate. |

In alternative preferred embodiments of the compositions of the first, second and third aspects of the present invention preferred embodiments of the compositions of the present invention the modified immediate release portion consists essentially of:

| | |
|---|---|
| (a) 80-90% | Guaifenesin; |
| (b) 3-6% | Dextromethorphan or a pharmaceutically acceptable salt |
| | thereof; |
| (c) up to 5% | Hypromellose; |
| (d) up to 2% | Hydroxy ethylcellulose; |
| (e) up to 5% | Microcrystalline cellulose; |
| (f) up to 1% | Magnesium stearate. |

In alternative preferred embodiments of the compositions of the first, second and third aspects of the present invention preferred embodiments of the compositions of the present invention the modified immediate release portion consists essentially of:

| | |
|---|---|
| (a) 80-90% | Guaifenesin; |
| (b) 3-6% | Dextromethorphan or a pharmaceutically acceptable salt |
| | thereof; |
| (c) up to 5% | Hypromellose; |
| (d) up to 5% | Hydroxy ethylcellulose; |
| (e) up to 5% | Microcrystalline cellulose; |
| (f) up to 1% | Magnesium stearate. |

In preferred embodiments of the compositions of the first, second and third aspects of the present invention the modified release portion consists essentially of:

| | |
|---|---|
| (a) 85-90% | Guaifenesin; |
| (b) 4-5% | Dextromethorphan or a pharmaceutically acceptable salt |
| | thereof; |
| (c) 3-6% | Hypromellose; |
| (d) 1-3% | Carbomer; |
| (e) 0.5-1% | Magnesium stearate. |

In preferred embodiments of the compositions of the first, second and third aspects of the present invention the modified release portion consists essentially of:

| | |
|---|---|
| (a) 90-93% | Guaifenesin; |
| (b) 4-6% | Dextromethorphan or a pharmaceutically acceptable salt |
| | thereof; |
| (c) 1-3% | Carbomer; |
| (d) 0.5-1% | Magnesium stearate. |

For the avoidance of doubt the present disclosure covers all possible combinations of the preferred embodiments of the immediate release analgesic-containing portion, the immediate release guaifenesin-containing portion and the modified release containing portion to give a composition having each of an immediate release analgesic-containing portion, an immediate release guaifenesin-containing portion and a modified release containing portion.

Modified release polymers that can be used in the compositions of the present invention include Acacia, Adipic Acid, Agar, Alginic Acid, Aliphatic Polyesters, Calcium Alginate, Carbomer, Carrageenan, Castor Oil, Cellaburate, Cellulose Acetate, Ceratonia, Colophony, Copovidone, Glyceryl Behenate, Glyceryl Monooleate, Glyceryl Monostearate, Glyceryl Palmitostearate, Hydroxypropyl Betadex, Hydroxypropyl Cellulose, Hydroxyethyl Cellulose, Hypromellose, Hypromellose Acetate Succinate, Methylcellulose, Polacrilin Potassium, Polycarbophil, Polydextrose, Polymethacrylates, Polyoxylglycerides, Polyvinyl Acetate Dispersion, Shellac, Sodium Alginate, Sodium Hyaluronate, Modified Starch, Sucrose Stearate, Microcrystalline Wax, White Wax, Yellow Wax, Xanthan Gum, Zein.

Hydrophilic polymers suitable for use in the sustained release portion include: one or more natural or partially or totally synthetic hydrophilic gums such as acacia, gum tragacanth, locust bean gum, guar gum, or karaya gum, modified cellulosic substances such as methylcellulose, hydroxymethylcellulose, hydroxypropyl methylcellulose, hydroxyethyl cellulose, hydroxypropyl cellulose, hydroxyethylcellulose, carboxyethylcellulose; proteinaceous substances such as agar, pectin, carrageen, and alginates; and other hydrophilic polymers such as carboxypolymethylene, gelatin, casein, zein, bentonite, magnesium aluminium silicate, polysaccharides, modified starch derivatives, and other hydrophilic polymers known to those of skill in the art or a combination of such polymers.

These hydrophilic polymers gel and dissolve slowly in aqueous acidic media thereby allowing the guaifenesin to diffuse from the gel in the stomach. When the gel reaches the intestines, it dissolves in controlled quantities in the higher pH medium, where the guaifenesin itself is fairly absorbable, to allow sustained release of guaifenesin throughout the digestive tract. Preferred hydrophilic polymers are the hydroxypropyl methylcelluloses such as those manufactured by The Dow Chemical Company and known as METHOCEL ethers. In one preferred embodiment of a sustained release formulation the hydrophilic polymer is a METHOCEL ether known as METHOCEL E10M.

Water-insoluble polymers which arc suitable for use in the sustained release portion are polymers which generally do not dissolve in solutions of a pH below 5, and dissolve more slowly in basic solutions than the hydrophilic polymer. Because the polymer is insoluble in low pH environments such as those found in gastric fluid, it aids in retarding drug release in those regions. Likewise, because the polymer dissolves more slowly in solutions of higher pH than hydrophilic polymers, it aids in retarding drug release throughout the intestines. This overall delayed release results in a more uniform serum concentration of guaifenesin.

The water-insoluble polymers suitable for use in the sustained release portion include: polyacrylic acids, acrylic resins, acrylic latex dispersions, cellulose acetate phthalate, polyvinyl acetate phthalate, hydroxypropyl methylcellulose phthalate, carbomer and other polymers common to those of skill in the art. In a preferred embodiment, a sustained release formulation comprises the acrylic resin CARBOPOL 974P supplied by BF Goodrich.

The sustained release portion of the present invention may further comprise pharmaceutical additives including, but not limited to: lubricants such as magnesium stearate, calcium stearate, zinc stearate, powdered stearic acid, hydrogenated vegetable oils, talc, polyethylene glycol, and mineral oil; colorants such as Emerald Green Lake and various FD&C colors; binders such as sucrose, lactose, gelatin, starch paste, acacia, tragacanth, povidone polyethylene glycol, Pullulan and corn syrup; glidants such as colloidal silicon dioxide and talc; surface active agents such as sodium lauryl sulfate, dioctyl sodium sulfosuccinate, tricthanolamine, polyoxyethylene sorbitan, poloxalkol, and quarternary ammonium salts; preservatives and stabilizers; excipients such as lactose, mannitol, glucose, fructose, xylose, galactose, sucrose, maltose, xylitol, sorbitol, chloride, sulfate and phosphate salts of potassium, sodium, and magnesium; and/or any other pharmaceutical additives known to those of skill in the art.

Colorants include, but are not limited to, Emerald Green Lake, FD&C Red #40, FD&C Yellow #6, FD&C Yellow #10, or FD&C Blue #1. In one preferred embodiment, a sustained release portion further comprises magnesium stearate and Emerald Green Lake. In another preferred embodiment, a sustained release formulation further comprises magnesium stearate and FD&C Blue #1 Aluminium Lake Dye.

The immediate release portion may comprise guaifenesin and various pharmaceutical additives such as disintegrants, lubricants, colorants, binders, glidants, surface active agents, preservatives, stabilizers, as described above and/or any other pharmaceutical additives known to those of skill in the art. Examples of suitable lubricant are as follows: Calcium Stearate, Glyceryl Behenate, Leucine, Magnesium Stearate, Mineral Oil, Myristic Acid, Palm Oil, Palmitic Acid, Poloxamer, Polyethylene Glycol, Potassium Benzoate, Sodium Benzoate, Sodium Lauryl Sulfate, Sodium Stearate, Sodium Stearyl Fumarate, Stearic Acid, Sucrose Stearate, Talc, Vegetable Oil, Zinc Stearate. Examples of suitable disintegrants are as follows: Carboxymethylcellulose Calcium, Carboxymethylcellulose Sodium, Sodium Lauryl Sulphate, Sodium Bicarboant, Chitosan, Coilloidal Sillicon Dioxide, Croscarmellose Sodium, Crospovidone, Glycine, Guar Gum, Lactose, Magnesium Aluminum Silicate, Polacrilin Potassium, Povidone, Sodium Alginate, Sodium Starch Glycolate. Examples of suitable diluents are as follows: Calcium Carbonate, Calcium Lactate, Calcium Phosphate, Calcium Silicate, Calcium Sulfate, Cellaburate, Cellulose Acetate, Microcrystalline Cellulose, Silicified Microcrystalline Cellulose, Corn Syrup Solids, Dextrates, Dextrin, Dextrose, Erythritol, Ethylcellulose, Fructose, Inulin, Isomalt, Kaolin, Lactitol, Lactose, Magnesium Carbonate, Magnesium Oxide, Maltitol, Maltodextrin, Maltose, Mannitol, Triglycerides, Polydextrose, Simethicone, Sodium Bicarbonate, Sodium Carbonate, Sodium Chloride, Sorbitol, Sucrose, Sugar, Sulfobutylether β-Cyclodextrin, Sunflower Oil, Talc, Trehalose, Xylitol. Examples of suitable binders are as follows: Attapulgite, Calcium Carbonate, Calcium Lactate, Ceratonia, Colophony, Copovidone, Ethylcellulose, Ethylene Glycol and Vinyl Alcohol Grafted Copolymer, Gelatin, Glucose, Hydroxethylmethyl Celluose, Magnesium Aluminium Silicate, Methylcellulose, Polycarbophil, Polydextrose, Polyethylene Oxide, Polymethacrylates, Povidone, Pullulan, Vitamin E Polyethylene Glycol Succinate.

For the avoidance of any doubt, reference to a pharmaceutically active compound includes all enantiomers and stereoisomers thereof, and also all pharmaceutically acceptable salts or esters thereof. For example, naproxen includes naproxen sodium, pseudoephedrine includes pseudoephedrine hydrochloride, dextromethorphan includes dextromethorphan hydrobromide.

In an alternative embodiment, the immediate release portion which comprises guaifenesin may further comprise the additional or more actives in the form of a drug/active-resin complex.

According to a fourth aspect of the present invention there is provided a sustained-release polymer matrix which consists essentially of a combination of a hydroxy propyl methyl cellulose having a molecular weight of 100,000-500,000 and a hydroxyethyl cellulose having a molecular weight of 500,000-2,000,000.

The molecular weight of hydroxyl propyl methyl cellulose can 200,000 to 300,000. A preferred molecular weight is 250,000.

The molecular weight of hydroxyethyl cellulose can 1,000,000 to 1,500,000. A preferred molecular weight is 1,300,000.

The ratio of the hydroxyl propyl methyl cellulose:hydroxyethyl cellulose is from 1:1 to 3:1. A preferred ratio is from 2:1 to 2.5:1. A more preferred ratio is 2.1:1.

According to a fifth aspect of the present invention there is provided the use of a sustained release polymer matrix as described in the fourth aspect in a pharmaceutical formulation.

The pharmaceutical formulation can comprise at least one active selected from an expectorant, an analgesic, an antihistamine, an antitussive, or a decongestant. The expectorant can be selected to be guaifenesin or n-acetyl cysteine. The analgesic can be selected to be naproxen, ketoprofen, diclofenac, ibuprofen and flurbiprofen. The antitussive can be selected to be dextromethorphan, codeine, codeine phosphate, codeine sulphate, diphenhydramine citrate, and diphenhydramine hydrochloride. The decongestant can be selected to be phenylephrine hydrochloride, phenylpropanolamine hydrochloride, pseudoephedrine or ephedrine. The antihistamine can be selected to be chlorpheniramine maleate, brompheniramine maleate, phenindamine tartrate, pyrilamine maleate, doxylamine succinate, phenyltoloxamine citrate, diphenhydramine hydrochloride, promethazine, and clemastine fumerate, or a combination thereof.

According to a sixth aspect of the present invention there is provided a method of providing relief from the symptoms of bronchial conditions, coughing and symptoms or diseases associated with coughing comprising administering to an individual a composition as described in the first, second or third aspects of the present invention.

According to a seventh aspect of the present invention there is provided the use of a composition as described in the first, second or third aspects of the present invention for the preparation of a medicament for the treatment of bronchial conditions, coughing and diseases associated with coughing.

According to an eighth aspect of the present invention these is provided an immediate release formulation of naproxen having an improved dissolution profile.

In a preferred embodiment the composition can comprise:

| | |
|---|---|
| (a) 50-52% | Naproxen or a pharmaceutically acceptable salt thereof; |
| (b) 35-40% | Microcrystalline cellulose; |
| (c) up to 5% | Povidone; |
| (d) 5-8% | Croscarmellose sodium; and |
| (e) up to 1% | Magnesium stearate. |

In a further preferred embodiment the composition can comprise:

| | |
|---|---|
| (a) 70-75% | Naproxen or a pharmaceutically acceptable salt thereof; |
| (b) 20-25% | Microcrystalline cellulose; |
| (c) up to 5% | Povidone; |
| (d) up to 1% | Magnesium stearate. |

In a further preferred embodiment the composition can comprise:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) 5-10% | Lactose; and |
| (f) up to 5% | Croscarmellose sodium. |

In a further preferred embodiment the composition can comprise:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) 5-10% | Lactose; and |
| (f) 5-10% | Croscarmellose sodium. |

In a further preferred embodiment the composition can comprise:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) up to 5% | Lactose; and |
| (f) 15-20% | Sodium Lauryl Sulphate. |

In a further preferred embodiment the composition can comprise:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) up to 2% | Croscarmellose sodium; and |
| (f) 15-20% | Sodium Lauryl Sulphate. |

In a further preferred embodiment the composition can consist essentially of:

| | |
|---|---|
| (a) 50-52% | Naproxen or a pharmaceutically acceptable salt thereof; |
| (b) 35-40% | Microcrystalline cellulose; |
| (c) up to 5% | Povidone; |
| (d) 5-8% | Croscarmellose sodium; and |
| (e) up to 1% | Magnesium stearate. |

In a further preferred embodiment the composition can consist essentially of:

| | |
|---|---|
| (a) 70-75% | Naproxen or a pharmaceutically acceptable salt thereof; |
| (b) 20-25% | Microcrystalline cellulose; |
| (c) up to 5% | Povidone; |
| (d) up to 1% | Magnesium stearate. |

In a further preferred embodiment the composition can consist essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) 5-10% | Lactose; and |
| (f) up to 5% | Croscarmellose sodium. |

In a further preferred embodiment the composition can consist essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) 5-10% | Lactose; and |
| (f) 5-10% | Croscarmellose sodium. |

In a further preferred embodiment the composition can consist essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) up to 5% | Lactose; and |
| (f) 15-20% | Sodium Lauryl Sulphate. |

In a further preferred embodiment the composition can consist essentially of:

| | |
|---|---|
| (a) 70-75% | Naproxen or a pharmaceutically acceptable salt thereof; |
| (b) 5-10% | Microcrystalline cellulose; |
| (c) up to 5% | Povidone; |
| (d) up to 1% | Magnesium stearate; |
| (e) up to 2% | Croscarmellose sodium; and |
| (f) 15-20% | Sodium Lauryl Sulphate. |

Typically, the composition has a dissolution profile of 68-70% after 10 mins and 93-98% after 20 mins when tested using US Pharmacopoeia apparatus 2 (paddles at 75 rpm at 37° C.) in Fessif media at pH 5.

Typically, the composition has a dissolution profile of 85-90% after 10 mins when tested using US Pharmacopoeia apparatus 2 (paddles at 75 rpm at 37° C.) in Fessif media at pH 6.8.

Fessif is a term used in the art to mean Fed State Simulated Intestinal Fluid. Fassif is a term used in the art to mean Fasted State Simulated Intestinal Fluid.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, the term “consisting essentially of” means the composition contains the indicated components and may contain additional components provided that the additional components that are non-active and do not materially affect the composition's basic characteristics. As used herein, the term “consisting of” means the composition contains the only indicated components and excludes other components.

As used herein, the term “up to” means a value greater than zero. For example, the term “up to 1%” means that component is present in the composition and can be present to an amount of 1%. The term “up to 5%” means that the component is present in the composition and can be present to an amount of 5%.

For the avoidance of doubt when the composition of the present invention is in the form of a tablet or tablets, the values given for both the ranges and amounts of the components in the compositions of the present invention refer to uncoated tablets. Additional coatings can be added as required.

Embodiments of the present invention will now be described by way of example only.

### Example 1

Tablet 1: Naproxen

| Ingredient | mg/tablet | | % Weight |
|---|---|---|---|
| Naproxen Sodium | 110.0 | mg | 73.33% |
| Microcrystalline Cellulose | 34.45 | mg | 22.96% |
| Povidone | 4.50 | mg | 3.0% |
| Mg Stearate | 1.05 | mg | 0.7% |
| Total Tablet | 150.0 | mg | 100.0% |

Tablet 2: Immediate/Sustained
Release Dextromethorphan and Guaifenesin

| Ingredient | mg/tablet | | % Weight |
|---|---|---|---|
| Guaifenesin | 600.0 | mg | 76.41% |
| Hypromellose | 50.00 | mg | 6.37% |
| MCC | 87.52 | mg | 11.15% |
| Dextromethorphan HBr | 30.0 | mg | 3.82% |
| Carbomer | 7.50 | mg | 0.96% |
| Sodium Starch Glycolate | 3.98 | mg | 0.51% |
| Colourant | 0.20 | mg | 0.025% |
| Mg Stearate | 6.0 | mg | 0.76% |
| Total Tablet | 785.2 | mg | 100.0% |

### Example 2

Tablet 1: Naproxen

| Ingredient | mg/tablet | | % Weight |
|---|---|---|---|
| Naproxen Sodium | 110.0 | mg | 51.16% |
| Microcrystalline Cellulose | 81.00 | mg | 37.67% |
| Crospovidone | 7.50 | mg | 3.49% |
| Croscarmellose sodium | 15.00 | mg | 6.98% |
| Mg Stearate | 1.50 | mg | 0.7% |
| Total Tablet | 215.0 | mg | 100.0% |

Tablet 2: Immediate/Sustained
Release Dextromethorphan and Guaifenesin

| Ingredient | mg/tablet | | % Weight |
|---|---|---|---|
| Guaifenesin | 600.0 | mg | 74.77% |
| Hypromellose | 19.00 | mg | 2.37% |
| MCC | 129.40 | mg | 16.12% |
| Dextromethorphan HBr | 30.0 | mg | 3.74% |
| Povidone | 7.00 | mg | 0.87% |
| Croscarmellose Sodium | 6.00 | mg | 0.74% |
| Hydroxy ethyl cellulose | 9.00 | mg | 1.12% |
| Colourant | 0.20 | mg | 0.025% |
| Mg Stearate | 1.9 | mg | 0.24% |
| Total Layer | 802.5 | mg | 100.0% |

### Example 3

Tablet 1: Naproxen

| Ingredient | mg/tablet | | % Weight |
|---|---|---|---|
| Naproxen Sodium | 110.0 | mg | 73.33% |
| Microcrystalline Cellulose | 14.55 | mg | 9.7% |
| Lactose | 12.65 | mg | 8.4% |
| Povidone | 4.65 | mg | 3.1% |
| Croscarmellose sodium | 7.15 | mg | 4.8% |
| Mg Stearate | 1 | mg | 0.7% |
| Total Tablet | 150.0 | mg | 100.0% |

Tablet 2: Immediate/Modified Release Dextromethorphan
and Guaifenesin Immediate Release Layer

| Ingredient | mg/tablet | | % Weight |
|---|---|---|---|
| Guaifenesin | 200 | mg | 63.77% |
| Dextromethorphan HBr | 10 | mg | 3.19% |
| Microcrystalline Cellulose | 85 | mg | 27.10% |
| Povidone | 10 | mg | 3.19% |
| Croscarmellose Sodium | 8.5 | mg | 2.71% |
| Mg Stearate | 0.15 | mg | 0.05% |
| Total Layer | 313.65 | mg | 100.00% |

Modified Release Layer

| Ingredient | mg/tablet | | % Weight |
|---|---|---|---|
| Guaifenesin | 400 | mg | 84.54% |
| Dextromethorphan HBr | 20 | mg | 4.23% |
| Hypromellose (K100M) | 24.5 | mg | 5.18% |
| Hydroxyethylcellulose | 12.25 | mg | 2.59% |
| Microcrystalline Cellulose | 14.75 | mg | 3.12% |
| Colourant | 0.1 | mg | 0.02% |
| Mg Stearate | 1.55 | mg | 0.33% |
| Total Layer | 473.15 | mg | 100.00% |
| Total Tablet | 786.8 | mg | 100.0% |

Tablet 1: Naproxen

| Ingredient | mg/tablet | | % Weight |
|---|---|---|---|
| Naproxen Sodium | 110 | mg | 73.33% |
| Microcrystalline Cellulose | 6 | mg | 4.00% |
| Lactose | 5 | mg | 3.33% |
| Povidone | 3 | mg | 2.00% |
| Sodium lauryl sulfate | 25 | mg | 16.67% |
| Mg Stearate | 1 | mg | 0.67% |
| Total Tablet | 150.0 | mg | 100.0% |

Tablet 2: Immediate/Modified Release
Dextromethorphan and Guaifenesin Immediate Release Layer

| Ingredient | mg/tablet | | % Weight |
|---|---|---|---|
| Guaifenesin | 200 | mg | 63.77% |
| Dextromethorphan HBr | 10 | mg | 3.19% |
| Microcrystalline Cellulose | 85 | mg | 27.10% |
| Povidone | 10 | mg | 3.19% |
| Croscarmellose Sodium | 8.5 | mg | 2.71% |
| Mg Stearate | 0.15 | mg | 0.05% |
| Total Layer | 313.65 | mg | 100.00% |

Modified Release Layer

| Ingredient | mg/tablet | | % Weight |
|---|---|---|---|
| Guaifenesin | 400 | mg | 84.54% |
| Dextromethorphan HBr | 20 | mg | 4.23% |
| Hypromellose (K100M) | 24.5 | mg | 5.18% |
| Hydroxyethylcellulose | 12.25 | mg | 2.59% |
| Microcrystalline Cellulose | 14.75 | mg | 3.12% |
| Colourant | 0.1 | mg | 0.02% |
| Mg Stearate | 1.55 | mg | 0.33% |
| Total Layer | 473.15 | mg | 100.00% |
| Total Tablet | 786.8 | mg | 100.0% |

Tablet 1: Naproxen

| Ingredient | mg/tablet | | % Weight |
|---|---|---|---|
| Naproxen Sodium | 110.0 | mg | 73.33% |
| Microcrystalline Cellulose | 14.55 | mg | 9.7% |
| Lactose | 12.65 | mg | 8.4% |
| Povidone | 4.65 | mg | 3.1% |
| Croscarmellose sodium | 7.15 | mg | 4.8% |
| Mg Stearate | 1 | mg | 0.7% |
| Total Tablet | 150.0 | mg | 100.0% |

Tablet 2: Immediate/Modified Release
Dextromethorphan and Guaifenesin Immediate Release Layer

| Ingredient | mg/tablet | | % Weight |
|---|---|---|---|
| Guaifenesin | 130 | mg | 54.83% |
| Dextromethorphan HBr | 7.00 | mg | 2.95% |
| Microcrystalline Cellulose | 85.00 | mg | 35.85% |
| Povidone | 8.00 | mg | 3.37% |
| Croscarmellose Sodium | 7.00 | mg | 2.95% |
| Mg Stearate | 0.10 | mg | 0.04% |
| Total Layer | 237.10 | mg | 100.00% |

Modified Release Layer

| Ingredient | mg/tablet | | % Weight |
|---|---|---|---|
| Guaifenesin | 470 | mg | 86.35% |
| Dextromethorphan HBr | 23 | mg | 4.23% |
| Hypromellose (K100M) | 17 | mg | 3.12% |
| Hydroxyethylcellulose | 17 | mg | 3.12% |
| Microcrystalline Cellulose | 15.5 | mg | 2.85% |
| Colourant | 0.1 | mg | 0.02% |
| Mg Stearate | 1.7 | mg | 0.31% |
| Total Layer | 544.3 | mg | 100.00% |
| Total Tablet | 781.40 | mg | 100.0% |

### Example 6

Tablet 1: Naproxen

| Ingredient | mg/tablet | | % Weight |
|---|---|---|---|
| Naproxen Sodium | 110 | mg | 73.33% |
| Microcrystalline Cellulose | 6 | mg | 4.00% |
| Lactose | 5 | mg | 3.33% |
| Povidone | 3 | mg | 2.00% |
| Sodium lauryl sulfate | 25 | mg | 16.67% |
| Mg Stearate | 1 | mg | 0.67% |
| Total Tablet | 150.0 | mg | 100.0% |

Tablet 2: Immediate/Modified
Release Dextromethorphan and Guaifenesin
Immediate Release Layer

| Ingredient | mg/tablet | | % Weight |
|---|---|---|---|
| Guaifenesin | 130 | mg | 54.83% |
| Dextromethorphan HBr | 7.00 | mg | 2.95% |
| Microcrystalline Cellulose | 85.00 | mg | 35.85% |
| Povidone | 8.00 | mg | 3.37% |
| Croscarmellose Sodium | 7.0 | mg | 2.95% |
| Mg Stearate | 0.10 | mg | 0.04% |
| Total Layer | 237.10 | mg | 100.00% |

Modified Release Layer

| Ingredient | mg/tablet | | % Weight |
|---|---|---|---|
| Guaifenesin | 470 | mg | 86.35% |
| Dextromethorphan HBr | 23 | mg | 4.23% |
| Hypromellose (K100M) | 17 | mg | 3.12% |
| Hydroxyethylcellulose | 17 | mg | 3.12% |
| Microcrystalline Cellulose | 15.5 | mg | 2.85% |
| Colourant | 0.1 | mg | 0.02% |
| Mg Stearate | 1.7 | mg | 0.31% |
| Total Layer | 544.3 | mg | 100.00% |
| Total Tablet | 781.40 | mg | 100.0% |

### Example 7

Tablet 1: Naproxen

| Ingredient | mg/tablet | | % Weight |
|---|---|---|---|
| Naproxen Sodium | 110 | mg | 73.33% |
| Microcrystalline Cellulose | 34.45 | mg | 22.97% |
| Povidone | 4.5 | mg | 3.00% |
| Mg Stearate | 1.05 | mg | 0.7% |
| Total Tablet | 150.0 | mg | 100.0% |

Tablet 2: Immediate/Modified Release Dextromethorphan and
Guaifenesin Immediate Release Layer

| Ingredient | mg/tablet | | % Weight |
|---|---|---|---|
| Guaifenesin | 85.5 | mg | 47.50% |
| Dextromethorphan HBr | 6.75 | mg | 3.75% |
| Microcrystalline Cellulose | 78.77 | mg | 43.76% |
| Hypromellose | 4.50 | mg | 2.50% |
| Sodium Starch Glycolate | 3.58 | mg | 1.99% |
| Mg Stearate | 0.90 | mg | 0.50% |
| Total Layer | 180.00 | mg | 100.00% |

Modified Release Layer

| Ingredient | mg/tablet | | % Weight |
|---|---|---|---|
| Guaifenesin | 514.50 | mg | 88.83% |
| Dextromethorphan HBr | 23.25 | mg | 4.03% |
| Hypromellose | 27.50 | mg | 4.75% |
| Carbomer | 8.25 | mg | 1.42% |
| Colourant | 0.22 | mg | 0.04% |
| Mg Stearate | 5.50 | mg | 0.95% |
| Total Layer | 579.22 | mg | 100.00% |
| Total Tablet | 759.22 | mg | 100.0% |

### Example 8

Tablet 1: Naproxen

| Ingredient | mg/tablet | | % Weight |
|---|---|---|---|
| Naproxen Sodium | 110 | mg | 73.33% |
| Microcrystalline Cellulose | 34.45 | mg | 22.97% |
| Povidone | 4.5 | mg | 3.00% |
| Mg Stearate | 1.05 | mg | 0.7% |
| Total Tablet | 150.0 | mg | 100.0% |

Tablet 2: Immediate/Modified Release Dextromethorphan and
Guaifenesin Immediate Release Layer

| Ingredient | mg/tablet | | % Weight |
|---|---|---|---|
| Guaifenesin | 76.00 | mg | 47.50% |
| Dextromethorphan HBr | 6.00 | mg | 3.75% |
| Microcrystalline Cellulose | 70.02 | mg | 43.76% |
| Hypromellose | 4.00 | mg | 2.50% |
| Sodium Starch Glycolate | 3.18 | mg | 1.99% |
| Mg Stearate | 0.80 | mg | 0.50% |
| Total Layer | 160.00 | mg | 100.00% |

Modified Release Layer

| Ingredient | mg/tablet | | % Weight |
|---|---|---|---|
| Guaifenesin | 424.00 | mg | 91.74% |
| Dextromethorphan HBr | 24.00 | mg | 5.19% |
| Carbomer | 9.90 | mg | 2.14% |
| Colourant | 0.26 | mg | 0.06% |
| Mg Stearate | 4.00 | mg | 0.87% |
| Total Layer | 462.16 | mg | 100.00% |
| Total Tablet | 622.16 | mg | 100.0% |

### Example 9

Tablet 1: Naproxen

| Ingredient | mg/tablet | | % Weight |
|---|---|---|---|
| Naproxen Sodium | 110 | mg | 73.33% |
| Microcrystalline Cellulose | 14.5 | mg | 9.67% |
| Povidone | 4.75 | mg | 3.17% |
| Mg Stearate | 0.75 | mg | 0.7% |
| Lactose | 10.00 | mg | 6.67% |
| Croscarmellose Sodium | 10.00 | mg | 6.67% |
| Total Tablet | 150.0 | mg | 100.0% |

Tablet 2: Immediate/Modified Release Dextromethorphan and
Guaifenesin Immediate Release Layer

| Ingredient | mg/tablet | | % Weight |
|---|---|---|---|
| Guaifenesin | 85.5 | mg | 47.50% |
| Dextromethorphan HBr | 6.75 | mg | 3.75% |
| Microcrystalline Cellulose | 78.77 | mg | 43.76% |
| Hypromellose | 4.50 | mg | 2.50% |
| Sodium Starch Glycolate | 3.58 | mg | 1.99% |
| Mg Stearate | 0.90 | mg | 0.50% |
| Total Layer | 180.00 | mg | 100.00% |

Modified Release Layer

| Ingredient | mg/tablet | | % Weight |
|---|---|---|---|
| Guaifenesin | 514.50 | mg | 88.83% |
| Dextromethorphan HBr | 23.25 | mg | 4.03% |
| Hypromellose | 27.50 | mg | 4.75% |
| Carbomer | 8.25 | mg | 1.42% |
| Colourant | 0.22 | mg | 0.04% |
| Mg Stearate | 5.50 | mg | 0.95% |
| Total Layer | 579.22 | mg | 100.00% |
| Total Tablet | 759.22 | mg | 100.0% |

### Example 10

Tablet 1: Naproxen

| Ingredient | mg/tablet | | % Weight |
|---|---|---|---|
| Naproxen Sodium | 110 | mg | 73.33% |
| Microcrystalline Cellulose | 14.5 | mg | 9.67% |
| Povidone | 4.75 | mg | 3.17% |
| Mg Stearate | 0.75 | mg | 0.7% |
| Lactose | 10.00 | mg | 6.67% |
| Croscarmellose Sodium | 10.00 | mg | 6.67% |
| Total Tablet | 150.0 | mg | 100.0% |

Tablet 2: Immediate/Modified Release Dextromethorphan and
Guaifenesin Immediate Release Layer

| Ingredient | mg/tablet | | % Weight |
|---|---|---|---|
| Guaifenesin | 76.00 | mg | 47.50% |
| Dextromethorphan HBr | 6.00 | mg | 3.75% |
| Microcrystalline Cellulose | 70.02 | mg | 43.76% |
| Hypromellose | 4.00 | mg | 2.50% |
| Sodium Starch Glycolate | 3.18 | mg | 1.99% |
| Mg Stearate | 0.80 | mg | 0.50% |
| Total Layer | 160.00 | mg | 100.00% |

Modified Release Layer

| Ingredient | mg/tablet | | % Weight |
|---|---|---|---|
| Guaifenesin | 424.00 | mg | 91.74% |
| Dextromethorphan HBr | 24.00 | mg | 5.19% |
| Carbomer | 9.90 | mg | 2.14% |
| Colourant | 0.26 | mg | 0.06% |
| Mg Stearate | 4.00 | mg | 0.87% |
| Total Layer | 462.16 | mg | 100.00% |
| Total Tablet | 622.16 | mg | 100.0% |

### Example 11

Tablet 1: Naproxen

| Ingredient | mg/tablet | | % Weight |
|---|---|---|---|
| Naproxen Sodium | 110 | mg | 72.37% |
| Microcrystalline Cellulose | 9.25 | mg | 6.09% |
| Povidone | 4.75 | mg | 3.13% |
| Mg Stearate | 1.00 | mg | 0.66% |
| Sodium Lauryl Sulphate | 25.00 | mg | 16.45% |
| Croscarmellose Sodium | 2.00 | mg | 1.32% |
| Total Tablet | 150.0 | mg | 100.0% |

Tablet 2: Immediate/Modified Release Dextromethorphan and
Guaifenesin Immediate Release Layer

| Ingredient | mg/tablet | | % Weight |
|---|---|---|---|
| Guaifenesin | 85.5 | mg | 47.50% |
| Dextromethorphan HBr | 6.75 | mg | 3.75% |
| Microcrystalline Cellulose | 78.77 | mg | 43.76% |
| Hypromellose | 4.50 | mg | 2.50% |
| Sodium Starch Glycolate | 3.58 | mg | 1.99% |
| Mg Stearate | 0.90 | mg | 0.50% |
| Total Layer | 180.00 | mg | 100.00% |

Modified Release Layer

| Ingredient | mg/tablet | | % Weight |
|---|---|---|---|
| Guaifenesin | 514.50 | mg | 88.83% |
| Dextromethorphan HBr | 23.25 | mg | 4.03% |
| Hypromellose | 27.50 | mg | 4.75% |
| Carbomer | 8.25 | mg | 1.42% |
| Colourant | 0.22 | mg | 0.04% |
| Mg Stearate | 5.50 | mg | 0.95% |
| Total Layer | 579.22 | mg | 100.00% |
| Total Tablet | 759.22 | mg | 100.0% |

### Example 12

Tablet 1: Naproxen

| Ingredient | mg/tablet | | % Weight |
|---|---|---|---|
| Naproxen Sodium | 110 | mg | 72.37% |
| Microcrystalline Cellulose | 9.25 | mg | 6.09% |
| Povidone | 4.75 | mg | 3.13% |
| Mg Stearate | 1.00 | mg | 0.66% |
| Sodium Lauryl Sulphate | 25.00 | mg | 16.45% |
| Croscarmellose Sodium | 2.00 | mg | 1.32% |
| Total Tablet | 150.0 | mg | 100.0% |

Tablet 2: Immediate/Modified Release Dextromethorphan and
Guaifenesin Immediate Release Layer

| Ingredient | mg/tablet | | % Weight |
|---|---|---|---|
| Guaifenesin | 76.00 | mg | 47.50% |
| Dextromethorphan HBr | 6.00 | mg | 3.75% |
| Microcrystalline Cellulose | 70.02 | mg | 43.76% |
| Hypromellose | 4.00 | mg | 2.50% |
| Sodium Starch Glycolate | 3.18 | mg | 1.99% |
| Mg Stearate | 0.80 | mg | 0.50% |
| Total Layer | 160.00 | mg | 100.00% |

Modified Release Layer

| Ingredient | mg/tablet | | % Weight |
|---|---|---|---|
| Guaifenesin | 424.00 | mg | 91.74% |
| Dextromethorphan HBr | 24.00 | mg | 5.19% |
| Carbomer | 9.90 | mg | 2.14% |
| Colourant | 0.26 | mg | 0.06% |
| Mg Stearate | 4.00 | mg | 0.87% |
| Total Layer | 462.16 | mg | 100.00% |
| Total Tablet | 622.16 | mg | 100.0% |

### Example 13—Composite Tablet

Immediate Release Layer

| Ingredient | mg/tablet | | % Weight |
|---|---|---|---|
| Guaifenesin | 140 | mg | 28.58% |
| Dextromethorphan HBr | 8 | mg | 1.63% |
| Naproxen Sodium | 110 | mg | 22.45% |
| Microcrystalline Cellulose | 60 | mg | 12.24% |
| Povidone | 11 | mg | 2.25% |
| Croscarmellose sodium | 26 | mg | 5.31% |
| Crospovidone | 9.8 | mg | 2.00% |
| Sodium lauryl sulfate | 25 | mg | 5.10% |
| Bicarbonate | 100 | mg | 20.41% |
| Mg Stearate | 0.1 | mg | 0.02% |
| Total Tablet | 489.9 | mg | 100.0% |

Modified Release Layer

| Ingredient | mg/tablet | | % Weight |
|---|---|---|---|
| Guaifenesin | 460 | mg | 84.73% |
| Dextromethorphan HBr | 22 | mg | 4.05% |
| Hypromellose | 28 | mg | 5.16% |
| Hydroxy ethyl cellulose | 14 | mg | 2.58% |
| Microcrystalline Cellulose | 17 | mg | 3.13% |
| Colourant | 0.1 | mg | 0.02% |
| Mg Stearate | 1.8 | mg | 0.33% |
| Total Layer | 542.9 | mg | 100.0% |

### Example 14—Composite Tablet

Immediate Release Layer

| Ingredient | mg/tablet | | % Weight |
|---|---|---|---|
| Guaifenesin | 200 | mg | 45.25% |
| Dextromethorphan HBr | 10 | mg | 2.26% |
| Naproxen Sodium | 110 | mg | 24.89% |
| Microcrystalline Cellulose | 45 | mg | 10.18% |
| Povidone | 11 | mg | 2.49% |
| Crospovidone | 65 | mg | 14.71% |
| Mg Stearate | 1 | mg | 0.23% |
| Total Tablet | 442 | mg | 100.0% |

Modified Release Layer

| Ingredient | mg/tablet | | % Weight |
|---|---|---|---|
| Guaifenesin | 400 | mg | 84.54% |
| Dextromethorphan HBr | 20 | mg | 4.23% |
| Hypromellose | 24.5 | mg | 5.18% |
| Hydroxy ethyl cellulose | 12.25 | mg | 2.59% |
| Microcrystalline Cellulose | 14.75 | mg | 3.12% |
| Colourant | 0.1 | mg | 0.02% |
| Mg Stearate | 1.55 | mg | 0.33% |
| Total Layer | 473.15 | mg | 100.0% |

### Example 15—Composite Tablet

Immediate Release Layer

| Ingredient | mg/tablet | | % Weight |
|---|---|---|---|
| Guaifenesin | 95 | mg | 24.26% |
| Dextromethorphan HBr | 7.5 | mg | 1.92% |
| Naproxen Sodium | 110 | mg | 28.09% |
| Microcrystalline Cellulose | 60 | mg | 15.32% |
| Povidone | 11 | mg | 2.81% |
| Croscarmellose sodium | 15 | mg | 3.83% |
| Crospovidone | 8.00 | mg | 2.00% |
| Sodium Lauryl Sulfate | 25 | mg | 5.10% |
| Sodium Bicarbonate | 60 | mg | 20.41% |
| Mg Stearate | 0.1 | mg | 0.03% |
| Total Layer | 391.6 | mg | 100.0% |

Modified Release Layer

| Ingredient | mg/tablet | | % Weight |
|---|---|---|---|
| Guaifenesin | 505 | mg | 89.35% |
| Dextromethorphan HBr | 22.5 | mg | 3.98% |
| Hypromellose | 25 | mg | 4.42% |
| Carbomer | 7.5 | mg | 1.33% |
| Colourant | 0.2 | mg | 0.04% |
| Mg Stearate | 5.0 | mg | 0.88% |
| Total Layer | 565.2 | mg | 100.0% |

### Example 16—Composite Tablet

Immediate Release Layer

| Ingredient | mg/tablet | | % Weight |
|---|---|---|---|
| Guaifenesin | 95 | mg | 27.34% |
| Dextromethorphan HBr | 7.5 | mg | 2.16% |
| Naproxen Sodium | 110 | mg | 31.65% |
| Microcrystalline Cellulose | 75 | mg | 21.58% |
| Povidone | 11 | mg | 3.17% |
| Croscarmellose sodium | 40 | mg | 11.51% |
| Crospovidone | 8.00 | mg | 2.30% |
| Mg Stearate | 1 | mg | 0.29% |
| Total | 347.5 | mg | 100.0% |

Modified Release Layer

| Ingredient | mg/tablet | | % Weight |
|---|---|---|---|
| Guaifenesin | 505 | mg | 89.35% |
| Dextromethorphan HBr | 22.5 | mg | 3.98% |
| Hypromellose | 25 | mg | 4.42% |
| Carbomer | 7.5 | mg | 1.33% |
| Colourant | 0.2 | mg | 0.04% |
| Mg Stearate | 5.0 | mg | 0.88% |
| Total Layer | 565.2 | mg | 100.0% |

### Example 17—Composite Tablet

Immediate Release Layer

| Ingredient | mg/tablet | | % Weight |
|---|---|---|---|
| Guaifenesin | 95 | mg | 24.32% |
| Dextromethorphan HBr | 7.5 | mg | 1.92% |
| Naproxen Sodium | 110 | mg | 28.16% |
| Microcrystalline Cellulose | 60 | mg | 15.36% |
| Povidone | 11 | mg | 2.82% |
| Croscarmellose sodium | 22 | mg | 5.63% |
| Sodium Lauryl Sulphate | 25.00 | mg | 6.40% |
| Sodium Bicarbonate | 60.00 | mg | 15.36% |
| Mg Stearate | 0.1 | mg | 0.03% |
| Total | 390.6 | mg | 100.0% |

Modified Release Layer

| Ingredient | mg/tablet | | % Weight |
|---|---|---|---|
| Guaifenesin | 505 | mg | 89.35% |
| Dextromethorphan HBr | 22.5 | mg | 3.98% |
| Hypromellose | 25 | mg | 4.42% |
| Carbomer | 7.5 | mg | 1.33% |
| Colourant | 0.2 | mg | 0.04% |
| Mg Stearate | 5.0 | mg | 0.88% |
| Total Layer | 565.2 | mg | 100.0% |

### Example 18—Composite Tablet

Immediate Release Layer

| Ingredient | mg/tablet | | % Weight |
|---|---|---|---|
| Guaifenesin | 95 | mg | 27.98% |
| Dextromethorphan HBr | 7.5 | mg | 2.21% |
| Naproxen Sodium | 110 | mg | 32.40% |
| Microcrystalline Cellulose | 75 | mg | 22.09% |
| Povidone | 11 | mg | 3.24% |
| Croscarmellose sodium | 40 | mg | 11.78% |
| Mg Stearate | 1 | mg | 0.29% |
| Total | 339.5 | mg | 100.0% |

Modified Release Layer

| Ingredient | mg/tablet | % Weight |
|---|---|---|
| Guaifenesin | 505 mg | 89.35% |
| Dextromethorphan HBr | 22.5 mg | 3.98% |
| Hypromellose | 25 mg | 4.42% |
| Carbomer | 7.5 mg | 1.33% |
| Colourant | 0.2 mg | 0.04% |
| Mg Stearate | 5.0 mg | 0.88% |
| Total Layer | 565.2 mg | 100.0% |

The tablets of the example embodiments of the present invention can be made using standard tableting procedures well-known to the person skilled in the art.

The bi-layer tablet may be manufactured according to any method known to those of skill in the art. The resulting tablet may comprise the two portions compressed against one another so that the face of each portion is exposed as either the top or bottom of the tablet, or the resulting tablet may comprise the sustained release portion in the centre coated by the immediate release portion so that only the immediate release portion is exposed. In a preferred embodiment, a bi-layer tablet of the present invention comprises the two portions compressed against one another so that the face of each portion is exposed.

The final dosage form can be made by inserting both the immediate-release naproxen tablet and the immediate-release/sustained or modified release guaifenesin/dextromethorphan tablet into a gelatin capsule which is then sealed. Furthermore, the composite tablets of Examples 3 and 4 are final dosage forms and need not, though they can, be inserted into a suitable capsule.

An advantage of the present invention is that there is provided a composition that provides multi-symptom cough/cold/flu relief for 12 hours. Currently, there are no other products that offer all of these significant benefits to consumers.

A further advantage is that there is provided a composition which comprises naproxen, guaifenesin and dextromethorphan in a single dose yet avoids any impact on the dissolution rate of either guaifenesin or dextromethorphan by naproxen.

The composition of the present invention also provides for more consistent release of both guaifenesin and dextromethorphan when compared to known products in various pH conditions and when exposed to various agitation speeds. For example purposes only, these novel formulations have been shown to improve the consistency of dextromethorphan release after 6 hours by 29% and guaifenesin release after 6 hours by 52% and 71%.

Further modifications and improvements can be made without departing from the scope of the invention described herein.

1. A pharmaceutical composition comprising a pharmaceutically effective amount of guaifenesin, naproxen and at least one further active which is selected to be an antitussive, a decongestant or an antihistamine.

2. (canceled)

3. (canceled)

4. A composition as claimed in claim 1, wherein the at least one further active is dextromethorphan.

5.-13. (canceled)

14. A composition as claimed in claim 1, wherein composition comprises immediate and sustained release portions.

15. (canceled)

16. (canceled)

17. A composition as claimed in claim 14, wherein the composition is provided with both sustained-release and immediate-release portions comprising guaifenesin.

18. (canceled)

19. A composition as claimed in claim 1, wherein the composition comprises:
(a) 50-85% Guaifenesin;
(b) up to 5% Dextromethorphan or a pharmaceutically acceptable salt thereof; and
(c) 5-30% Naproxen or a pharmaceutically acceptable salt thereof.

20.-54. (canceled)
